(19) United States
(12) Patent Application Publication
(10) Pub. No.: US 2012/0294832 A1
(43) Pub. Date: Nov. 22, 2012

(54) COMPOSITION COMPRISING INTERFERON ALPHA

(75) Inventors: Gauthier Pouliquen, Lyon (FR); You-Ping Chan, Ternay (FR); Rémi Meyrueix, Lyon (FR); David Chognot, Serpaize (FR); Roger Kravtzoff, Algans (FR)

(73) Assignee: Flamel Technologies, Venissieux (FR)

(21) Appl. No.: 13/472,675

(22) Filed: May 16, 2012

Related U.S. Application Data
(60) Provisional application No. 61/488,513, filed on May 20, 2011.

(30) Foreign Application Priority Data
May 20, 2011 (FR) FR 11 54416
May 16, 2012 (IB) PCT/IB2012/052459

(51) Int. Cl. A61K 38/21

(52) U.S. Cl. 424/85.7

(57) ABSTRACT

The present invention relates to a novel solid composition, useful for treating hepatitis, in particular hepatitis C, comprising at least one interferon alpha and at least one grafted poly(glutamic acid) having an average molar mass ranging from 26,000 to 40,000 g/mol, preferably approximately 33,000 g/mol and carrying grafts of alpha-tocopherol at an average molar grafting rate ranging from 4.5 to 5.5%, preferably approximately 5%, the interferon alpha and said grafted poly(glutamic acid) being present in a grafted poly(glutamic acid)/interferon alpha weight ratio ranging from 21 to 125.

It also relates to the use of such a solid composition for the preparation of a liquid composition by the addition of an aqueous liquid.

The present invention aims to propose a novel pharmaceutical composition of interferon alpha (IFN-alpha). IFN-alpha is indicated in the treatment of leukaemias, lymphomas, melanomas and certain other cancers as well as hepatitis B and C.

Hepatitis C is an infectious disease caused by the hepatitis C virus (HCV), characterized by inflammation of the liver, which can develop into chronic hepatitis, and subsequently cirrhosis and cancer of the liver. According to the World Health Organisation estimates, approximately 170 million people worldwide are affected by chronic infection with hepatitis C virus, and 3 to 4 million people are newly infected every year.

Treatments for combating chronic HCV infection are aimed at eradicating the HCV, in other words a significant and lasting reduction in the viral load. This is generally evaluated by the “sustained virologic response” (SVR) which characterizes the absence of detection of the HCV RNA, six months after stopping the treatment.

Initially, the treatment of hepatitis C involved the sub-cutaneous injection of unmodified IFN-alpha. However, because of the short life of unmodified IFN-alpha in the plasma, approximately ten hours, the treatment required repeated injections, of the order of three times a week. Such a treatment was accompanied by significant side effects, due to the “saw-tooth” profile of the IFN-alpha concentration in the plasma, characterized by the succession of peaks of high IFN-alpha concentration in the plasma, each followed by a rapid decrease.

For the purposes of improving the IFN-alpha plasma profile, pegylated forms (PEG-IFN-alpha) have been developed: the conjugation of IFN-alpha with polyethylene glycol leads to an improvement in the bioavailability of the IFN-alpha, in particular an increase in the duration of life in the plasma, and as a result advantageously makes it possible to reduce the frequency of administration. Nowadays, the reference treatment for chronic HCV infection thus consists of a weekly injection of pegylated IFN-alpha.

By way of example, the products utilizing modified IFN-alpha, in particular PEG-IFN-alpha-2a (for example, the product marketed under the trade name Pegasys® by Laboratoires Roche) or PEG-IFN-alpha-2b (for example, the products marketed under the trade names Viraferon® Peg and PEG-Intron® by Laboratoires Schering-Plough) can be administered once a week, instead of the three injections required for standard IFN-alpha.

However, the pegylation of IFN-alpha, which involves the formation of a covalent bond between the polyethylene glycol (PEG) and the IFN-alpha molecule with a PEG chain configuration, either linear (PEG-IFN-alpha-2b) or branched (PEG-IFN-alpha-2a), creates a steric hindrance, leading to a lowering of the antiviral efficacy of IFN-alpha. It has thus been shown that the PEG-IFN-alpha-2b retained only 28% of its activity in comparison with an unmodified IFN-alpha-2b (Caliceti, 2004, Pharmacokinetics of pegylated interferons, Dig Liver Dis 36, S334-339). It follows that it is necessary to increase the dose administered for effective treatment of chronic hepatitis C.

Unfortunately, the treatment of hepatitis C with significant doses of IFN-alpha may be accompanied by a certain number of undesirable side effects, which currently constitute one of the major concerns in the management of patients infected with HCV. These undesirable effects are most often characterized by depressive symptoms, anxiety, mood and behaviour disorders (aggression, impulsiveness, irritability, emotional psychosis) as well as neutropenia. These side effects are considered a major obstacle to the initiation of treatment, a cause of poor compliance, or even a definitive stopping of anti-hepatitis C treatment. They can thus lead the practitioner to limit the dose of IFN-alpha administered, thus compromising the chances of eradication of the virus and survival of the patient.

A need therefore exists for novel compositions having fewer undesirable side effects, and a sustained duration of action comparable to that of the pegylated IFN-alpha compositions.

A composition utilizing IFN-alpha must moreover meet a certain number of essential requirements.

First of all, the preparation protocol of such a composition must not, for obvious reasons, lead to a degradation or denaturation of the IFN-alpha. Degradation could for example occur during a rise in temperature, through the use of surfactants, by bringing the IFN-alpha into contact with an organic solvent, or by high shearing. It is therefore particularly important that the IFN-alpha can be utilized in an aqueous process requiring no excessive temperature, no surfactant, nor any organic solvent.

IFN-alpha moreover has a tendency to form dimers and aggregates of greater mass, which can render its compositions immunogenic (Diress A. et al., J. Chromatogr. A, 2010, 1217(19): 3297-3306; and Ruiz L. et al., Int. J. Pharm., 2003, 264(1-2): 57-72). It is therefore particularly desirable to have a composition protecting the IFN-alpha against the formation of such aggregates.

Moreover, it is indispensable that the IFN-alpha composition, as presented at the time of use by the patient, has a low viscosity in order to allow easy parenteral administration, in particular sub-cutaneous injection, through a needle with a small diameter, for example 0.4 mm (gauge 27 G).

On the other hand, it is desirable that the composition exhibits good stability over time.

Finally, within the context of hepatitis treatment, it is necessary for the IFN-alpha thus formulated to be able to be released over a period of the order of a week, while maintaining sufficient activity and bioavailability.

The development of an IFN-alpha composition making it possible to overcome the drawbacks of the compositions currently available, and meeting all of the abovementioned requirements is difficult to achieve.

The present invention aims precisely to propose novel compositions utilizing interferon alpha. The term “interferon alpha” (IFN-alpha) as used in the description is meant to denote leukocyte interferons, lymphoblast interferons or interferon alpha, in particular of human origin, obtained either by the extraction of biological fluids or by techniques utilizing recombinant DNAs, as well as their muteins, their salts, their functional derivatives, their variants, their analogues and their active fragments. More particularly, the interferon alpha according to the invention is chosen from interferon alpha-2b (IFN alpha-2b) and interferon alpha-consensus (IFN alpha-consensus).

The present invention according to a first of its aspects, relates to a solid composition comprising at least one interferon alpha (IFN-alpha) and at least one grafted poly(glutamic acid) having an average molar mass ranging from 26,000 to 40,000 g/mol, preferably from 28,000 to 38,000 g/mol, preferably approximately 33,000 g/mol, and carrying grafts of alpha-tocopherol at an average molar grafting rate ranging from 4.5 to 5.5%, preferably approximately 5%, the IFN-alpha and said grafted poly(glutamic acid) being present in a grafted poly(glutamic acid)/IFN-alpha weight ratio ranging from 21 to 125.

According to a second aspect, the present invention relates to an aqueous liquid composition comprising at least one IFN-alpha and at least one grafted poly(glutamic acid) having an average molar mass ranging from 26,000 to 40,000 g/mol preferably from 28,000 to 38,000 g/mol, preferably approximately 33,000 g/mol, and carrying grafts of alpha-tocopherol at an average molar grafting rate ranging from 4.5 to 5.5%, the IFN-alpha and said grafted poly(glutamic acid) being present in a grafted poly(glutamic acid)/IFN-alpha weight ratio ranging from 21 to 125, said composition being obtained by the addition of an aqueous liquid to a solid composition as defined previously.

According to yet another of its aspects, the present invention relates to a solid pharmaceutical composition based on IFN-alpha comprising a solid composition as defined previously.

By “pharmaceutical”, is meant a composition intended for treating humans or animals.

According to another of its aspects, the invention relates to the use of such a solid pharmaceutical composition for preparing a liquid pharmaceutical composition.

According to another of its aspects, the invention relates to the method for preparing a solid composition as defined previously.

The solid composition according to the invention can moreover comprise at least one antioxidant.

It can also comprise at least one lyoprotectant.

It can finally comprise one or more excipients, known to a person skilled in the art, making it possible to adjust in particular the osmolality and the pH of the composition.

Different preferred variants of compositions according to the invention are described below.

The uses of polymers in the field of the formulation of active proteins are numerous.

Thus, De Leede et al., in J. Interferon Cytokine Res. (2008) 28: 113-122, describe a controlled-release formulation of IFN-alpha-2b contained in microspheres (20 to 50 μm) of poly(ether-ester). These poly(ether-ester)s are more particularly poly(ethylene glycol) and poly(butylene terephthalate) block copolymers as described in U.S. Pat. No. 5,980,948.

The application WO 03/104303 discloses poly(aspartic acid)s and poly(glutamic acid)s, partially grafted with alpha-tocopherol. These polymers spontaneously form a colloidal suspension of hydrogels with nanometric size in water. These hydrogels are capable of combining for example with proteins, more particularly insulin.

The international application WO 2010/100220 proposes prodrugs constituted by IFN-alpha bound to a water-soluble polymeric carrier by an auto-cleavable bond, in particular of carbamate or amide type, making it possible to release the IFN-alpha with a half-life greater than 4 days.

Finally, from the teaching of the document WO 2005/051417, fluid aqueous colloidal suspensions for the sustained release of interferon are known, comprising the non-covalent combination of the IFN-alpha with submicronic particles of a poly(glutamic acid) grafted by hydrophobic groups. It is in particular stressed that, in order to have a sustained release duration, the polymer concentration must be high enough, and greater than a critical concentration C1, in order to allow the formation of a gelled deposit after parenteral injection. The critical concentration C1 is determined in vitro by a test described in detail in document WO 2005/051417.

The present inventors have found that such a system, which is particularly advantageous in terms of duration of action, proves nevertheless to be capable of improvement. Thus, the liquid compositions given as examples exhibit insufficient stability in the long term and therefore do not make it possible to envisage storage over a long period of time. Moreover, when the concentration of grafted poly(glutamic acid) is too great, the bioavailability of the IFN-alpha is thereby reduced.

The present invention has in particular the advantage of compensating for the abovementioned limitations.

Thus, the solid composition according to the invention advantageously protects the IFN-alpha from the phenomena of aggregation, as demonstrated in Examples 3 and 9 which follow. An aqueous liquid composition according to the invention has a percentage of IFN-alpha in the monomeric form (i.e. non-aggregated) greater than 80%, preferably greater than 90%, preferably even greater than 95% of the total quantity of IFN-alpha.

The solid composition according to the invention it exhibits very good stability, in particular over at least two years at 5° C., as demonstrated in Example 5 which follows.

The solid composition according to the invention has a low viscosity as illustrated in Examples 2 and 8, in order to allow easy parenteral administration, in particular sub-cutaneous injection, through a needle with a small diameter, for example 0.4 mm (gauge 27 G)

A composition according to the invention is such that the period for releasing in vitro 60% of the IFN-alpha is greater than 120 minutes, preferably greater than 150 minutes, according to the in vitro test T1 described below.

A composition according to the invention is such that the period for releasing in vitro 60% of the IFN-alpha is greater than 100 minutes, preferably greater than 120 minutes, according to the in vitro test T2 described below.

A composition according to the invention can also make it possible to ensure sustained in vivo release of the IFN-alpha with an effective life time in the plasma, in particular over a duration of at least 5 days, in particular of at least 7 days, while maintaining sufficient activity and bioavailability. Such a sustained release of the IFN-alpha makes it possible to envisage administration on a weekly basis, which is more advantageous for the patient.

More particularly, a composition according to the invention advantageously allows a significant and lasting reduction in the viral load. As demonstrated in Example 17, the reduction in the viral load is comparable, in particular at least equal, to that obtained with a pegylated IFN-alpha.

On the other hand, the inventors have found that an IFN-alpha composition, more particularly of IFN-alpha-2b or of IFN-alpha-consensus, and of grafted polymer as defined above, in particular in a polymer/IFN-alpha weight ratio according to the invention, makes it possible to reduce the undesirable side effects. This composition thus opens up the possibility of treating the patients for whom treatment with pegylated IFN-alpha proved difficult to tolerate and sometimes ineffective.

Finally, the stability of the solid composition of the invention as well as the simplicity of its preparation method make it possible to envisage its commercial development.

Thus, the solid composition according to the invention can be advantageously packaged in dosage units adapted to the preparation, generally extemporaneous, of an injectable pharmaceutical dose by the addition of an aqueous liquid, in particular of water for injection.

The IFN-alpha concentration in the solid composition can be advantageously adjusted in order to allow for the reconstitution of a liquid composition in an injectable dose, in particular with an IFN-alpha concentration ranging from 0.2 to 0.8 mg/mL.

The quantities of solid composition and aqueous liquid can also be adjusted so that said liquid composition has a grafted poly(glutamic acid) concentration ranging from 17 to 25 mg/mL.

Other characteristics, advantages and embodiments of the composition according to the invention will become more apparent on reading the description which follows.

In the remainder of the text, the expressions “comprised between . . . and . . . ”, “ranging from . . . to . . . ” and “varying from . . . to . . . ” are equivalent and are meant to signify that the limits are inclusive, unless otherwise specified.

Unless otherwise indicated, the expressions “containing a” and “comprising a” should be understood as “containing at least one” and “comprising at least one”.

Within the meaning of the invention, the term “approximately” means that the value which follows this term is verified taking account of the limits of experimental error acceptable to a person skilled in the art.

Solid Composition

Interferon Alpha

The IFN-alpha utilized in a composition of the invention is more particularly IFN-alpha-2b or IFN alpha-consensus.

IFN-alpha-2b is a protein belonging to the family of type I interferons, known for its antiviral activity in the treatment of active chronic hepatitis C.

IFN-alpha-consensus is a protein the amino acid sequence of which is that in which each amino acid is that most frequently encountered in the corresponding position in the different sequences of the natural IFN-alpha sub-types.

IFN-alphas are commercially available, in solution in an appropriate buffer.

By way of example, there can be mentioned the solution of IFN-alpha-2b presented in a buffer containing citric acid (25 mM), dibasic sodium phosphate (50 mM) and sodium chloride (150 mM), stored frozen, marketed by BioSidus, Argentina.

As an example of a commercially available solution of IFN-alpha-consensus there can be mentioned the solution of IFN-alpha-consensus interferon alfacon-1, presented in a buffer containing sodium chloride (100 mM), monobasic sodium phosphate (9.6 mM) and dibasic sodium phosphate (17.4 mM) and stored at +5° C., marketed by the company Three Rivers Pharmaceuticals under the name Infergen®.

Poly(Glutamic Acid) Grafted with Alpha-Tocopherol

A composition according to the invention utilizes at least one poly(glutamic acid) grafted with alpha-tocopherol, at a rate of 21 to 125 mg, per 1 mg of IFN-alpha.

The grafted poly(glutamic acid) utilized according to the invention has an average molar mass ranging from 26,000 to 40,000 g/mol, preferably comprised between 28,000 and 38,000 g/mol, and more preferentially approximately 33,000 g/mol.

Moreover it has an average molar grafting rate with alpha-tocopherol ranging from 4.5 to 5.5%, preferably approximately 5%.

The poly(glutamic acid) has a linear backbone, or main chain, formed by glutamic acid or glutamate units.

The poly(glutamic acid) utilized according to the invention is more particularly a homopolymer of alpha-poly(glutamic acid) type, corresponding to the structure (i) below:

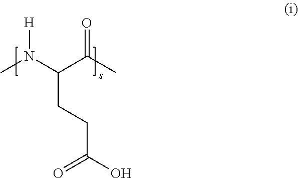

The alpha-poly(glutamic acid) of the invention is of L, D or racemic (D,L) configuration.

The residual carboxylic functions of the grafted alpha-poly(glutamic acid) are either neutral (—COOH glutamic acid form), or ionized (—COO⁻ glutamate anion), depending on the pH and the composition. In the second case, the neutrality of the polymer requires the presence of a counter-ion which can be an inorganic cation, for example sodium.

In aqueous solution, in particular during the reconstitution of a solution by the addition of water to the solid composition as described below, more generally at a pH comprised between 6 and 8, the polymer is mainly in the form of poly(glutamate).

Alpha-poly(glutamic acids) which can be used for implementing the invention are commercially available, in particular under reference 386847 from Sigma-Aldrich®. They can also be synthesized by polymerization of N-carboxyamino acid anhydrides (NCA), described, for example, in the article “Biopolymers, 1976, 15, 1869” and in the work by H. R. Kricheldorf “alpha-Aminoacid-N-carboxy Anhydride and related Heterocycles”

Springer Verlag (1987). These polymers can also be synthesized according to the route described in the patent application FR 2 801226.

The alpha-tocopherol utilized can be presented in its D-alpha-tocopherol form (its natural form), its L-alpha-tocopherol form or its D,L-alpha-tocopherol form (“all-racemic” and synthetic form).

The alpha-tocopherol according to the invention is preferably of synthetic origin.

According to a particularly preferred embodiment, the grafted alpha-poly(glutamic acid) does not comprise grafts other than the alpha-tocopherol type grafts.

Preferably, the grafted alpha-poly(glutamic acid) according to the invention corresponds to general formula (I) below:

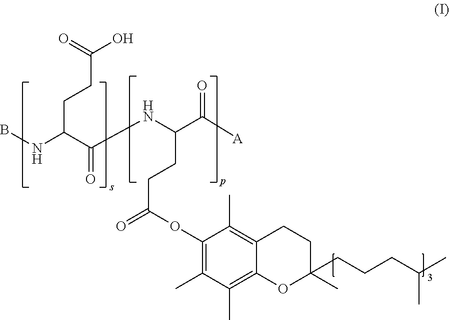

in which:

A represents independently:
an NHR group in which R represents a hydrogen, a linear C₂ to C₁₀ alkyl, a branched C₃ to C₁₀ alkyl or a benzyl,
or
a terminal amino acid unit bound by nitrogen;

B represents a hydrogen, a linear C₂ to C₁₀ acyl, a branched C₃ to C₁₀ acyl or a pyroglutamate;

p corresponds to the average number of glutamate monomers carrying an alpha-tocopheryl group;

s corresponds to the average number of non-grafted glutamate monomers. The average molar grafting rate of the poly(glutamic acid) with tocopherol groups p/(s+p) is the ratio between the average number of monomers grafted with alpha-tocopherol and the total number of monomers, and is comprised between 4.5 and 5.5%, preferably equal to approximately 5%.

The average degree of polymerization DP=s+p is comprised between 180 and 250, preferably between 200 and 240 and in particular equal to approximately 220.

The grafted alpha-poly(glutamic acid) of the invention can be obtained by methods known to a person skilled in the art, in particular synthesized by means of one of the methods described in the Applicant's international patent application WO 03/104303, in particular the method described below.

The coupling of the alpha-tocopherol with some of the carboxylic functions of the alpha-poly(glutamic acid) is carried out by reaction of said polymer with alpha-tocopherol in the presence of a coupling agent and a catalyst in a suitable solvents such as dimethylformamide (DMF), N-methyl-pyrrolidone (NMP) or dimethylsulphoxide (DMSO). The grafting rate is chemically controlled by the stoichiometry of the constituents and reagents or by the reaction time.

The grafted alpha-poly(glutamic acid) utilized according to the invention is capable, when it is dispersed in an aqueous liquid with a pH ranging from 6 to 8, in particular water, of spontaneously forming hydrogels of nanometric size, capable of combining non-covalently with IFN-alpha.

The terms “combine”, “combination” or “combined”, used to describe the relationships between interferon alpha and the polymer as utilized in the invention, signify that interferon alpha is combined with the polymer by non-covalent physical interactions, in particular hydrophobic, electrostatic interactions, hydrogen bonds, or via a steric encapsulation by said polymers.

Preferably, the grafted alpha-poly(glutamic acid) utilized in the present invention has a L configuration. It is biodegradable, in particular degraded in approximately 10 days after injection into rats and in approximately 15 days after injection into dogs.

Determination of the Average Molar Mass, of the Average Molar Grafting Rate with Alpha-tocopherol and of the Degree of Polymerization of the Grafted Poly(Glutamic Acid)

The average molar mass within the meaning of the invention is defined by the peak molar mass (Mp) measured by size exclusion chromatography.

In order to measure its average molar mass, the polymer sample is precipitated by the addition of 0.1 N hydrochloric acid, freeze-dried then dissolved in N-methyl-pyrrolidone (NMP).

The average peak molar mass is measured by means of a size exclusion chromatography equipment comprising 3 sequential polystyrene-co-divinylbenzene chromatography columns (5 μm/100,000 Å, 5 μm/10,000 Å and 5 μm/1,000 Å) conditioned in N-methyl-pyrrolidone. This equipment is coupled to an 18 angle static light scattering detector (e.g. DAWN EOS—Wyatt Technology) and a differential refractometer (e.g. OptiLab REX—Wyatt Technology), which enables to determine absolute molar masses.

The molar grafting rate of alpha-tocopherol in grafted poly(glutamic acid) corresponds to the difference between the measured total alpha-tocopherol rate and the measured free alpha-tocopherol rate. The total alpha-tocopherol rate is determined by ¹H NMR, while the free alpha-tocopherol rate is determined by HPLC. The free alpha-tocopherol rate is determined by HPLC by means of a microBondapak C18 column (300 mm long, 3.9 mm of internal diameter, filled with 10 microns diameter spherical silica) provided by Waters or similar, conditioned at 40° C. and eluted in isocratic mode with the mobile phase, comprising 25% vol methanol and 75% vol acetonitrile with a 1 mL/min flow rate.

The total alpha-tocopherol rate is determined by ¹H NMR with a 300 MHz NMR spectrophotometer (Avance 300) equipped with a QNP probe. The polymer sample is freeze-dried, dissolved in deuterated trifluoroacetic acid, then analyzed.

Two signals are considered
the signal around 0.6 ppm corresponds to the protons of the 4 methyl groups of the alpha-tocopherol aliphatic chain
the signal around 4.5-4.7 ppm corresponds to the proton in the a position of the glutamic acid unit.

Each signal is integrated and the integral value corresponding to the proton in a-position of the glutamic acid unit is calibrated to 100.

The molar fraction in monomer units grafted with alpha-tocopherol groups can thus be deduced.

The molar grafting rate of alpha-tocopherol corresponds to the molar fraction x₂ in monomer units grafted with alpha-tocopherol groups. The molar fraction x₁ in non-grafted monomer units can thus be deduced.

The average degree of polymerization DP is calculated by dividing the average molecular weight Mₚ of a polymer chain determined by size exclusion chromatography as described above, by the average molecular weight M of a monomer unit of the polymer: DP=Mₚ/M.

This average molecular weight of a unit is the average of the molecular weights of the units constituting the polymer, each being weighted by the molar fraction of this unit.

Considering M₁ the average molecular weight of the non-grafted glutamic acid monomers, and M₂ the average molecular weight of glutamic acid monomers grafted with alpha-tocopherol. The average weight M is given by the following formula: M=x₁·M₁+x2·M2.

Compositions

According to a first embodiment variant, the IFN-alpha utilized is IFN-alpha-2b. A composition of the invention can then more particularly comprise IFN-alpha-2b and said grafted poly(glutamic acid) according to the invention, in a grafted poly(glutamic acid)/IFN alpha-2b weight ratio ranging from 54 to 100, preferably from 60 to 90.

According to a second embodiment variant, the IFN-alpha utilized is IFN-alpha-consensus. A composition of the invention can then more particularly comprise IFN-alpha-consensus and said grafted poly(glutamic acid) according to the invention, in a grafted poly(glutamic acid)/IFN alpha-consensus weight ratio ranging from 30 to 60, preferably from 40 to 50.

A composition according to the invention can comprise, apart from the IFN-alpha and grafted poly(glutamic acid), one or more excipients, in particular at least one antioxidant, at least one lyoprotective excipient and other excipients known to a person skilled in the art, in particular used for adjusting the osmolality and pH of the composition.

Antioxidant

According to a particularly preferred embodiment, a composition according to the invention comprises at least one antioxidant.

The antioxidant used is for example methionine, cysteine, ascorbic acid, an ascorbate, citric acid or a citrate.

In particular, a composition of the invention can comprise at least one antioxidant in an antioxidant/IFN-alpha weight ratio ranging from 2 to 7.

According to a first embodiment variant, the IFN-alpha utilized is IFN-alpha-2b. A composition of the invention can then comprise methionine in a methionine/IFN-alpha-2b weight ratio ranging from 4 to 6.4.

According to a second embodiment variant, the IFN-alpha utilized is IFN-alpha-consensus. A composition of the invention can then comprise methionine in a methionine/IFN-alpha-consensus weight ratio ranging from 2.5 to 3.5.

The methionine utilized according to the invention is more particularly L-methionine.

Lyoprotective Excipient

According to a particularly preferred embodiment, a composition of the invention can comprise at least one lyoprotective excipient, for example a sugar, polyvinylpyrrolidone or polyethylene glycol.

By “sugar”, is meant simple sugars (small molecules composed of one or two carbohydrate units) or complex sugars (long chains of carbohydrate units), but also polyols in general.

As examples, there can be mentioned lactose, glucose, fructose, sucrose, mannitol, xylitol, erythritol, the sorbitols, trehaloses, and mixtures thereof

Preferably, the sugar is more particularly chosen from sucrose, mannitol and mixtures thereof

As examples of mannitol, there can be mentioned the different grades of Roquette's Pearlitol®, in particular Pearlitol® SD200.

In particular, a composition of the invention can comprise one or more lyoprotective excipients in a lyoprotective excipient/IFN alpha weight ratio ranging from 50 to 500.

According to a first embodiment variant, the IFN-alpha utilized is IFN-alpha-2b. A composition of the invention can then comprise at least one sugar in a sugar/IFN-alpha-2b weight ratio ranging from 110 to 280.

According to a second embodiment variant, the IFN-alpha utilized is IFN-alpha-consensus. A composition of the invention can then comprise at least one sugar in a sugar/IFN-alpha-consensus weight ratio ranging from 70 to 160.

Excipients for Adjustment of the Osmolality and pH

By “osmolality”, is meant a measurement of the number of osmoles (i.e. of the moles of particles in solution) of solute, per kg of water. It can be measured using a Fiske osmometer (mark 3).

The excipients used for adjusting the osmolality of the composition include for example sodium chloride, potassium chloride, lactose, glucose, fructose, sucrose, mannitol, xylitol, erythritol, sorbitol, trehalose, maltodextrin, and mixtures thereof

The excipients used for adjusting the pH of the composition include for example sodium hydroxide, acetic acid and hydrochloric acid.

According to a first embodiment variant, a solid composition according to the invention comprises the following constituents:
IFN-alpha-2b;
a grafted poly(glutamic acid) according to the invention in a grafted poly(glutamic acid)/IFN-alpha-2b weight ratio ranging from 54 to 100, preferably from 60 to 90;
methionine in a methionine/IFN-alpha-2b weight ratio ranging from 4 to 6.4; and
one or more sugar(s), in particular chosen from sucrose and/or mannitol, in a sugar(s)/IFN-alpha-2b weight ratio ranging from 110 to 280.

A particularly preferred solid composition according to the invention, comprising IFN-alpha-2b, comprises the following constituents:
a) approximately 0.3 mg of IFN-alpha-2b;
b) approximately 22 mg of grafted poly(glutamic acid) as described previously;
c) approximately 1.5 mg of methionine; and
d) approximately 53 mg of sucrose;
or a multiple or sub-multiple of said quantities.

According to a second embodiment variant, a solid composition according to the invention comprises the following constituents:
IFN-alpha-consensus;
a grafted poly(glutamic acid) according to the invention in a grafted poly(glutamic acid)/IFN-alpha-consensus weight ratio ranging from 30 to 60, preferably from 40 to 50;
methionine in a methionine/IFN-alpha-consensus weight ratio ranging from 2.5 to 3.5; and
one or more sugar(s) in a sugar(s)/IFN-alpha-consensus weight ratio ranging from 70 to 160.

A particularly preferred solid composition, comprising IFN-alpha-consensus, comprises the following constituents:
a) approximately 0.45 mg of IFN-alpha-consensus;
b) approximately 20 mg of grafted poly(glutamic acid) as described previously;
c) approximately 1.5 mg of methionine; and
d) approximately 58 mg of sucrose;
or a multiple or sub-multiple of said quantities.

Apart from the IFN-alpha, the grafted poly(glutamic acid), the antioxidant and the lyoprotectant, a composition according to the invention can comprise one or more excipients. The choice of these excipients is clearly within the competence of a person skilled in the art.

Method for the Preparation of the Solid Composition Comprising IFN-alpha and Grafted Poly(Glutamic Acid)

According to a particular embodiment the present invention relates to a method for the preparation of a solid composition comprising IFN-alpha-2b, comprising the following stages:

(a) providing an aqueous liquid solution of grafted poly(glutamic acid) at a concentration comprised between 20 and 30 mg/g;

(b) providing an IFN-alpha-2b solution at a concentration comprised between 1.5 and 2.7 mg/mL, preferably comprised between 2.0 and 2.2 mg/mL, more preferentially at a concentration of approximately 2.1 mg/mL; with at least one component selected from a lyoprotective excipient, an antioxidant and a pH adjustment excipient, being present in at least one of the solutions of stage (a) or stage (b);

(c) mixing the solutions of stages (a) and (b) so that after mixing, the composition obtained:
has a grafted poly(glutamic acid) concentration comprised between 10 and 15 mg/g,
has an osmolality comprised between 130 and 230 mOsm/kg,
has a pH comprised between 6.2 et 6.8, preferably 6.5;

(d) subjecting said mixture to at least one sterilization operation; and

(e) dehydrating the solution in order to form said solid composition.

According to a second particular embodiment, the present invention relates to a method for the preparation of a solid composition comprising IFN-alpha-consensus, comprising the following stages:

(a) providing a solution of grafted poly(glutamic acid) at a concentration comprised between 20 and 30 mg/g;

(b) providing a solution of IFN-alpha-consensus at a concentration comprised between 2.0 and 5.0 mg/mL, preferably 3.4 mg/mL; with at least one component selected from a lyoprotective excipient, an antioxidant and a pH adjustment excipient, being present in at least one of the solutions of stage (a) or stage (b);

(c) mixing the solutions of stages (a), (b) and (c) so that after mixing, the composition :
has a grafted poly(glutamic acid) concentration comprised between 10 and 15 mg/g;
has an osmolality comprised between 130 and 250 mOsm/kg,
has a pH comprised between 6.8 and 7.2, preferably 7.0;

(d) subjecting said mixture to at least one sterilization operation; and

(e) dehydrating the solution in order to form said solid composition.

According to a particular embodiment of these methods, stage (d) is performed by sterilizing filtration and comprises several filtration stages, advantageously two filtration stages separated by stirring for at least 2 hours at ambient temperature. In particular, the filters used have a pore diameter of 0.2 microns and are for example Supor EKV, Supor DCF500 or AcroCap Supor filters equipped with a polyethersulphone membrane marketed by Pall.

The solution obtained at the end of stage (d) is dehydrated during stage (e) by a standard dehydration method such as lyophilization, atomization or evaporation, preferably by lyophilization.

According to a particular embodiment, the solution is distributed into flasks or vials before being dehydrated during stage (e).

According to another particular embodiment, the flasks or vials contain glass beads.

Liquid Composition

The solid compositions described above make it possible, by the addition of a solvent such as water, to prepare the corresponding liquid compositions. They make it possible, for example by the addition of water for injection (WFI), to prepare injectable liquid compositions which are ready for use.

A subject of the invention is also an aqueous liquid composition, in particular obtained by the addition of an aqueous liquid to a solid composition as defined previously, more particularly injectable, comprising, in an aqueous liquid, at least:
an IFN-alpha at a concentration comprised between 0.2 and 0.8 mg/mL;
a grafted poly(glutamic acid) having an average molar mass ranging from 26,000 to 40,000 g/mol and carrying grafts of alpha-tocopherol at an average molar grafting rate ranging from 4.5 to 5.5%,

the interferon alpha and said grafted poly(glutamic acid) being present in a grafted poly(glutamic acid)/IFN-alpha weight ratio ranging from 21 to 125.

According to a preferred embodiment, the aqueous liquid composition has a grafted poly(glutamic acid) concentration according to the invention ranging from 17 to 25 mg/mL.

Of course, a person skilled in the art is capable of adjusting the solid composition and aqueous liquid quantities utilized in order to obtain a liquid aqueous composition having the desired IFN-alpha and grafted poly(glutamic acid) concentrations.

According to a particular embodiment, the aqueous liquid composition reconstituted from the solid composition according to the invention has a pH comprised between 6.3 and 7.4.

According to another particular embodiment, the reconstituted aqueous liquid composition has an osmolality comprised between 270 and 350 mOsmol/kg.

According to a first embodiment variant, when the IFN-alpha is IFN-alpha-2b, the liquid aqueous pharmaceutical composition according to the invention can have an IFN-alpha-2b concentration ranging from 0.27 to 0.33 mg/mL, preferably approximately 0.3 mg/mL.

Within the context of this variant, the liquid aqueous composition of the invention can more particularly have from 19 to 25 mg/mL, preferably approximately 22 mg/mL, of grafted poly(glutamic acid).

A particularly preferred aqueous liquid composition, comprising IFN-alpha-2b, comprises the following constituents:
a) approximately 0.3 mg of IFN-alpha-2b;
b) approximately 22 mg of grafted poly(glutamic acid) as described previously;
c) approximately 1.5 mg of methionine; and
d) approximately 53 mg of sucrose;
or a multiple or sub-multiple of said quantities.

Such a composition preferably has a pH of approximately 6.5 and an osmolality of approximately 300 mOsm/kg.

According to another embodiment variant, when the IFN-alpha is IFN-alpha-consensus, the liquid aqueous pharmaceutical composition according to the invention can more particularly have an IFN-alpha-consensus concentration ranging from 0.40 to 0.50 mg/mL, preferably approximately 0.45 mg/mL.

Within the context of this variant, the liquid aqueous composition of the invention can more particularly have 17 to 23 mg/mL of grafted poly(glutamic acid), in particular approximately 20 mg/mL.

A particularly preferred aqueous liquid composition, comprising IFN-alpha-consensus, comprises the following constituents:
a) approximately 0.45 mg of IFN-alpha-consensus;
b) approximately 20 mg of grafted poly(glutamic acid) as described previously;
c) approximately 1.5 mg of methionine; and
d) approximately 58 mg of sucrose;
or a multiple or sub-multiple of said quantities.

Such a composition preferably has a pH of approximately 7.0 and an osmolality of approximately 300 mOsm/kg.

According to another particularly advantageous embodiment, the reconstituted aqueous liquid composition has a viscosity, measured at 20° C. and with a shear gradient of 10 s⁻¹, less than 1,000 mPa·s, preferably less than 500 mPa·s, in particular ranging from 5 to 200 mPa·s.

The viscosity can be measured at 20° C., using an AR1000 type rheometer (TA instrument) on which a cone-plane type geometry has been installed (4 cm and 2° angle), for a shear gradient of 10 s⁻¹.

Advantageously, the reconstituted aqueous liquid composition is thus suitable for parenteral administration, in particular sub-cutaneous injection through a needle with a small diameter, for example 0.4 mm (gauge 27 G).

Preferably, the aqueous liquid composition according to the invention is presented in the form of an aqueous suspension of hydrogels, of nanometric size, in particular with an average hydrodynamic diameter by volume comprised between 10 and 60 nm.

Protocols and Methods

Determination of the Size of the “Hydrogels”

The average hydrodynamic diameter of the hydrogels is measured by dynamic light scattering according to well-known methods, for example by means of a Malvern zeta sizer nano-ZS device or ALV CGS 3 equipment. In the case of the latter, the scattering angle is 140°.

In order to carry out the measurement, the solid composition is dissolved in water so as to obtain a polymer concentration of 22 mg/mL. A 0.15 M NaCl solution is added so as to obtain a polymer concentration of 1 mg/mL. The mixture is maintained under moderate stirring for 24 hours, then filtered through two sequential filters with respective pore sizes of 0.8 and 0.2 μm before being analyzed by dynamic light scattering at a pH comprised between 6 and 7.

The scattering signal acquisition time is 10 minutes. The measurement is carried out three times on two samples. The result is the average of the 6 measurements.

Preferably, the hydrogels have an average volume diameter ranging from 10 to 60 nm, in particular from 10 to 30 nm, and more particularly ranging from 10 to 20 nm.

Method for the Evaluation of IFN-alpha Aggregation

In order to demonstrate the protection against aggregation provided by the composition according to the invention, the samples are heated at 90° C. for one hour.

The quantity of aggregates in the composition can be evaluated by the following two methods:
size exclusion chromatography (SEC) in the presence of sodium dodecyl sulphate (SDS)
“Western blot”.

Measurement of the Quantity of IFN-alpha in the Monomeric Form by SDS-SEC:

The quantity of IFN-alpha in monomeric form, in other words of non-aggregated IFN-alpha, is evaluated by comparison of the composition according to the invention with a range of standard IFN-alphas surrounding the concentration aimed at.

The samples and the standards are then diluted with a 2% sodium dodecyl sulphate (SDS) solution then injected into a TSK G4000 SW×1 column. The mobile phase is a 3.3 mM PBS solution comprising 0.3% SDS.

The quantity of IFN-alpha in the monomeric form is evaluated by comparison of the intensity of the peak eluted for the composition according to the invention with the peaks corresponding to the standards in the IFN-alphas range.

Evaluation of the Presence of Aggregates by Western Blot

Stage 1:

A sample of 200 ng of composition according to the invention is diluted in Laemmli buffer at pH 6.8 (2% SDS, 62.4 mM Tris(hydroxymethyl)-aminomethane buffer solution, 0.06% bromophenol blue, 10% glycerol) then deposited on a 12% polyacrylamide gel. The grafted poly(glutamic acid), the IFN-alpha and any aggregates are separated by electrophoresis. The running buffer is a <<XT MES Running buffer>> (Biorad—Ref 161 0789) containing between 1 and 2.5% SDS.

Stage 2:

After migration of the proteins in the gel, the different forms of IFN-alpha (monomer and aggregates) are transfected onto a nitrocellulose membrane. The IFN-alpha is then specifically revealed using an anti-IFN-alpha primary antibody and a secondary antibody coupled with alkaline phosphatase. The membrane is then stained with a mixture of 5-Bromo-4-Chloro-3′-Indolyphosphate, p-Toluidine and nitroblue tetrazolium (NBT) chloride.

After staining, the IFN-alpha and its aggregates appear as well-separated stained bands. Direct visual comparison of the intensity of the bands makes it possible to determine whether or not the tested composition contains aggregates.

### Characterization of the In-Vitro Release Profile of the Compositions According to the Invention.

Each of the compositions to be tested is injected into a medium of controlled porosity in which circulation of an eluent containing albumin is established. At different times, the eluent is collected and the quantity of IFN-alpha in the different fractions is measured by an immunoassay, by an ELISA method (Immunotech, Beckman Coulter, kit Réf IM3193) or by an ECLIA method (Meso Scale Discovery, kit Ref K151ACA-4), depending on the supplier's instructions.

For each of the samples, the test is carried out three times. Depending on the experiment, either version described hereinafter can be used.

In-Vitro Test T1:

The test is carried out by means of a SOTAX 3239 type continuous flow cell with a diameter of 22.6 mm, filled with 1 mm diameter glass beads (SOTAX F200-0110) in its lower part and with an inert material of controlled porosity (Carpenter RP 30263 polyurethane foam, height 30 mm, diameter 25 mm, supplied by SEDES CREATION) in the upper part.

The eluent is prepared by dissolving powder of bovine serum albumin (BSA—albumin fraction V, VWR, 1.120.18.0100) at a concentration of 30 g/L in 10 mM phosphate buffered saline PBS (Sigma, P4417) containing L-methionine (Degussa GmBH, 1101660818) at a concentration of 10 mM.

After closing the cell, a continuous flow of 5 mL/min of eluent is applied using a peristaltic pump (IPC8, SOTAX, M300-0008), the flow cell being maintained at 37° C. in a water bath. After balancing the system and stopping the pump, the cell is opened and 50 μL of the sample to be tested is injected into the foam at a depth of 0.5 cm using a 100 μL syringe equipped with a 50 mm needle (Fisher Bioblock A23310). The syringe is weighed before and after injection in order to determine precisely the quantity of sample injected.

The cell is closed again and an eluent flow rate of 0.5 mL/min is applied continuously through the cell. On leaving the cell, the liquid is collected at regular intervals using a fraction collector (FC 204, GILSON, 171041). The samples are stored at +4° C.

In-Vitro Test T2:

The test is carried out by means of a SOTAX 3239 type continuous flow cell with a diameter of 22.6 mm, filled with 1 mm diameter glass beads (SOTAX F200-0110) in its lower part. The eluent is prepared by dissolving powder of bovine serum albumin (BSA -albumin fraction V, VWR, 1.120.18.0100) at a concentration of 30 g/L in 10 mM phosphate buffered saline PBS (Sigma, P4417).

After closing the cell, a continuous flow of 5 mL/min of eluent is applied using a peristaltic pump (IPC8, SOTAX, M300-0008), the flow cell being maintained at 37° C. in a water bath. After balancing the system and stopping the pump, the cell is opened and a filtration plate of 20 mm diameter, with a porosity 0 (160-250 nm, VWR, 511-0020) impregnated with 200 μL of the sample to be tested is deposited onto a bed of glass beads. The filtration plate is weighed before and after injection in order to determine precisely the quantity of sample impregnated.

The cell is closed again and an eluent flow rate of 0.5 mL/min is applied continuously through the cell. On leaving the cell, the liquid is collected at regular intervals using a fraction collector (FC 204, GILSON, 171041). The samples are stored at +4° C.

The invention will be better understood and its advantages and embodiment variants will become clearly apparent from the examples and figures which follow, given only by way of illustration.

### FIGURES

FIG. 1 represents the plasma concentration of IFN-alpha-2b released as a function of time, wherein:

[symbol] represents the concentration of IFN-alpha-2b released from composition

B2,

[symbol] represents the concentration of IFN-alpha-2b released from the control solution,

- - - represents the limit of quantification of IFN-alpha-2b.

FIG. 2 represents the plasma concentration of IFN-alpha-consensus released as a function of time, wherein:

[symbol] represents the concentration of IFN-alpha-consensus released from composition D2,

[symbol] represents the concentration of IFN-alpha-consensus released from the control solution,

- - - represents the limit of quantification of IFN-alpha-consensus.

FIG. 3 represents the plasma concentration of IFN-alpha-2b released as a function of time, wherein:

[symbol] represents the concentration of IFN-alpha-2b released from composition K,

[symbol] represents the concentration of IFN-alpha-2b released from the composition of Viraferon®Peg.

### EXAMPLES

### Example 1

Synthesis of the Grafted Poly(Glutamic Acid) Polymer P1 Used in the Compositions According to the Invention

A grafted poly(glutamic acid) with a DP of approximately 220 and grafted at approximately 5 molar % in alpha-tocopherol is prepared according to the following protocol.

15 g of a alpha-poly(L-glutamic acid) with a DP of approximately 220 is solubilized in 288 mL of dimethylformamide (DMF) at 80° C. The mixture is cooled down to 15° C. and 2.5 g of all-racemic alpha-tocopherol (>98% obtained from Fluka®) solubilized in 8 mL of DMF, 280 mg of 4-dimethylaminopyridine solubilized in 1 mL of DMF and 1.6 g of diisopropylcarbodiimide solubilized in 6 mL of DMF are added successively. After 3.5 hours under stirring, the reaction medium is neutralized with an aqueous solution of soda. The polymer is then purified by ultrafiltration on a 1 kDa membrane and concentrated to approximately 30 mg/mL. The solution is filtered on a 0.22 μm membrane, and stored at 5° C. before use.

The average peak molar mass (Mp) measured using an 18-angle light scattering detector (MALLS) coupled with size exclusion chromatography equipment is 31,000 g/mol. The level of grafted alpha-tocopherol, estimated by proton NMR spectroscopy, is 5.1 molar %.

### Example 2

Preparation and Characterization of an Aqueous Liquid Composition According to the Invention (Composition B1), Reconstituted from a Solid Composition (Composition A) According to the Invention, Containing Polymer P1 and IFN-alpha-2b

Stage 1: Preparation of the Initial Liquid Composition

498 g of water for injection, 9.2 g of a 1 N acetic acid solution, 239 g of a sucrose solution at 300 mg/g and 51 g of a methionine solution at 40 mg/g are added successively to a solution of 1,014 g of polymer P1 at 29.9 mg/mL. The solution of polymer P1 obtained is maintained under moderate stirring for 2 hours at 25° C.

A frozen solution of IFN-alpha-2b at 2.3 mg/mL (BioSidus—Argentina) in a buffer containing citric acid (25 mM), dibasic sodium phosphate (50 mM) and sodium chloride (150 mM) is thawed at ambient temperature.

161 g of this solution is added to 1,642 g of the solution of polymer P1. The mixture thus obtained is left under stirring at 25° C. for 2 hours before being sterilized by filtration on a 0.2 μm membrane then left to rest for another 16 hours. The mixture is then divided, at a rate of 1.31 g per flask, into 3 mL flasks each containing 3 glass beads with a diameter of 4.76 mm.

Stage 2: Preparation of a Solid Composition A According to the Invention

The mixture obtained in stage 1, divided into the flasks, is then lyophilized in a USIFROID freeze-dryer with a freeze-drying cycle lasting a total of 72 hours, in order to obtain a solid composition A according to the invention.

Stage 3: Reconstitution of an Aqueous Liquid Composition B1 According to the Invention

An injectable aqueous liquid composition B1 is reconstituted by the addition of 0.85 mL of water for injection per flask and stirred manually for a few minutes. The liquid composition B1 has a grafted poly(glutamic acid)/IFN-alpha-2b weight ratio of 77 and contains 53 mg/mL of sucrose and 1.5 mg/mL of L-methionine.

The characteristics of composition B1 are shown in Table 1.

TABLE 1
Characteristics of liquid composition B1

| | Composition B1 |
|---|---|
| C_{IFN} (mg/mL) | 0.29 |
| Cₚₒₗ (mg/mL) | 22.4 |
| [polymer P1]/[IFN-alpha-2b] (g/g) | 77 |
| pH | 6.6 |
| Osmolality (mOsm/kg) | 291 |
| Viscosity (mPa · s) | 79 |
| Hydrodynamic diameter (nm) | 20 |

### Example 3

Measurement of the Percentage of Aggregation of IFN-alpha-2b in the Liquid Composition B1 According to the Invention

A liquid composition B1 is prepared as described in Example 2.

A control solution of IFN-alpha-2b at 0.3 mg/mL is prepared by diluting in water for injection a freshly thawed, concentrated solution of IFN-alpha-2b, as described in Example 2.

Evaluation of the Aggregation of IFN Alpha-2b

i—Visually

Visual inspection shows that the two solutions are clear.

0.9 mL of composition B1 and 0.9 mL of a control solution of IFN-alpha-2b are heated for 1 hour at 90° C.

At the end of this heating process, it appears clearly that composition B1 is still clear whereas the control solution is cloudy, indicating the aggregation of some of the IFN-alpha-2b in the latter.

ii—By UV Absorbance

This difference in behaviour is confirmed by measurement of the UV absorbance at 450 nm of composition B1 and the control solution of IFN-alpha-2b on a Perkin Elmer Lambda 35 spectrophotometer equipped with cells with a width of 3 mm, as shown in Table 2.

TABLE 2
UV absorbance at 450 nm of the solutions before and after heating

| | Composition B1 | |
|---|---|---|
| | (according to the | Control solution of IFN- |
| | invention) | alpha-2b |
| Absorbance before heating | 0.005 | 0.03 |
| (absorbance unit) | | |
| Absorbance after heating | 0.005 | 0.34 |
| (absorbance unit) | | |

The low absorbance of composition B1 after heating indicates that no aggregation of IFN-alpha-2b occurred.

iii—By Size Exclusion Chromatography

Composition B1 and the control solution of IFN-alpha-2b, before and after heating for 1 hour at 90° C., are analyzed by size exclusion chromatography.

The results are shown in Table 3.

TABLE 3
Percentage of IFN alpha-2b monomer

| | Composition B1 | Control |
|---|---|---|
| | (according to the | solution of IFN- |
| | invention) | alpha-2b |
| peak of IFN-alpha-2b monomer | 100% | 97% |
| before heating (surface %) | | |
| peak of IFN-alpha-2b monomer | 97% | 58% |
| after heating (surface %) | | |

These results show that more than 95% of the IFN-alpha-2b is eluted as IFN-alpha-2b monomer in the case of the heated composition B1, whereas this peak represents no more than 58% in the case of the heated control solution of IFN-alpha-2b.

iv—Western Blot Method

The samples of composition B1 and of a control solution of IFN-alpha-2b are analyzed Western Blot. Analysis of the membrane reveals the presence of aggregates in the case of the control solution of IFN-alpha-2b heated for 1 hour at 90° C., as well as a reduction in the quantity of IFN-alpha-2b monomer with respect to the non-heated control solution. In the case of the heated sample of composition B1, no aggregates are observed and the quantity of IFN-alpha-2b monomer is identical to that of the non-heated composition B1.

In conclusion, these different tests clearly show that a composition according to the invention makes it possible to prevent the aggregation of IFN-alpha-2b.

### Example 4

Pharmacokinetic Properties in the Dog of an Aqueous Liquid Composition According to the Invention (composition B2), Containing Interferon Alpha-2B, Reconstituted Before Injection From a Solid Composition According to the Invention

Preparation of Composition B2

A liquid composition B2 is prepared as described in Example 2. The liquid composition B2 has a grafted poly(glutamic acid)/IFN-alpha-2b weight ratio of 71 and contains 53 mg/mL of sucrose and 1.5 mg/mL of L-methionine.

The characteristics of this composition are presented in Table 4.

TABLE 4
Characteristics of composition B2

| | Composition B2 |
|---|---|
| C_{IFN} (mg/mL) | 0.31 |
| Cₚₒₗ (mg/mL) | 22.0 |
| [polymer P1]/[IFN-alpha-2b] (g/g) | 71 |
| pH | 6.7 |
| Osmolality (mOsm/kg) | 284 |

Pharmacokinetic Properties of Composition B2

Composition B2 is injected by sub-cutaneous route into four Beagle dogs weighing approximately 10 kg, in the dorsal position, at a dose of 60 μg of IFN-alpha-2b per kg (i.e. 0.2 mL of composition per kg).

A control solution of IFN-alpha-2b is prepared at a concentration of 0.3 mg/mL in a solution of phosphate buffered saline (PBS—Sigma) containing 10 mM of monobasic sodium phosphate, 2.7 mM of potassium chloride and 137 mM of sodium chloride. This control solution is also tested on four other Beagle dogs according to a protocol similar to that described above.

The blood samples for determining the plasma concentrations of IFN-alpha-2b are taken at the following times: T=0 hours and 1 hour, 6 hours, 12 hours, 18 hours, 24 hours, 36 hours, 48 hours, 72 hours, 96 hours, 120 hours, 144 hours, 168 hours and 240 hours after injection. The serum samples are frozen at −80° C.

Analysis of these samples is then carried out by an ELISA method with a commercial kit (EIA IFNα, ref. IM3193, Immunotech/Beckman Coulter) using IFN-alpha-2b as standard and following the supplier's instructions.

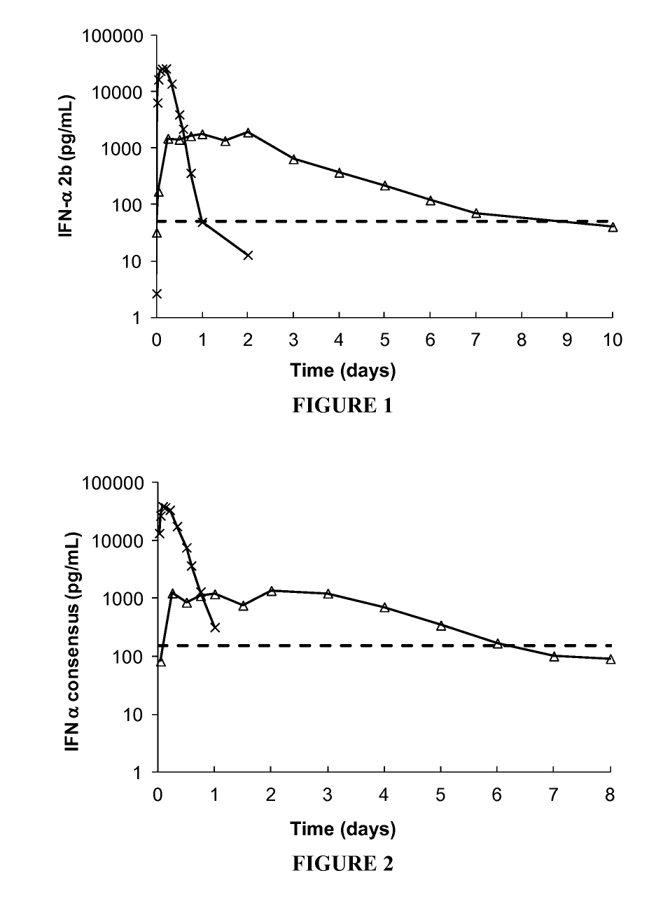

As shown in FIG. 1, the plasma profile of IFN-alpha-2b released from composition B2 stretches over a period of at least 5 days while the plasma profile of IFN-alpha-2b released from the control solution cannot be quantified anymore after only one day.

The following pharmacokinetic parameters were calculated using the WinNonlin software non-compartmental analysis (NCA) method (WinNonlin Professional v5.1, Pharsight Corporation), excepted the T_{50% AUC} which was calculated using Excel software (Microsoft):
Cₘₐₓ, Tₘₐₓ: the maximum serum concentration (Cmax) and time (Tmax) taken to reach this Cmax, by direct reading of the concentration points;
AUC₀₋ₜ: the area under the curve of concentration as a function of time between the time t₀ (pre-dose) and the last time measured, calculated by the trapezoid rule;
T_{50% AUC}: the time taken to reach 50% of the AUC, by linear interpolation.

The results are shown in Table 5.

TABLE 5
Pharmacokinetic parameters corresponding to composition B2 and to
a control solution of IFN-alpha-2b

| | Control | Composition B2 |
|---|---|---|
| | solution of | (according to the |
| | IFN-alpha-2b | invention) |
| Cₘₐₓ ± standard deviation (ng/mL) | 27.5 ± 7.7 | 2.2 ± 0.6 |
| median Tₘₐₓ [interval] (h) | 3 [2-5] | 21 [12-48] |
| AUC₀₋ₜ ± standard deviation | 211.3 ± 34.4* | 121.1 ± 36.5** |
| (ng × h/mL) | | |
| T_{50%AUC} ± standard deviation (h) | 5.2 ± 0.6 | 44.0 ± 5.1 |

*AUC calculated between 0 and 48 h
**AUC calculated between 0 and 120 h

A reduction in the Cₘₐₓ and a significant extension of the Tₘₐₓ and of the T_{50% AUC} for composition B2 were observed in comparison with a control solution of IFN-alpha-2b administered at the same concentration, which demonstrates the sustained release properties of a composition according to the invention combining IFN-alpha-2b with a grafted poly(glutamic acid).

### Example 5

Compared Stability of a Solid Composition According to the Invention and a Non-Lyophilized Composition

A solid composition A is prepared as described in Example 2.

On the other hand, a liquid composition is obtained according to a method of preparation analogous to that described in stage 1 of Example 2, by mixing a solution of polymer P1 and a solution of IFN-alpha-2b, without the addition of sucrose solution or methionine solution, in order to obtain a mixture having a polymer P1 concentration of 22 mg/mL and an IFN-alpha-2b concentration of 0.3 mg/mL.

Flasks containing the solid composition A and flasks containing the liquid composition thus prepared are placed in a chamber maintained at 5° C.

The flasks are removed at different time intervals and analyzed with HPLC with an elution gradient, the phases being composed of a water/acetonitrile +0.2% TFA mixture (phase A 70/30; phase B 20/80). The degraded forms are evaluated by measurement of their surface percentage.

The results obtained for the two solutions are shown in Table 6.

TABLE 6
Degraded forms (surface %) as a function of time

| | | Composition A |
|---|---|---|
| | | (according to the |
| Time | Liquid composition | invention) |
| T₀ | 2.2 | 1.1 |
| 2 months | 3.5 | 1.7 |
| 6 months | 5.1 | 1.7 |
| 9 months | 6.1 | 1.6 |
| 12 months | 7.3 | 1.2 |
| 18 months | 10.7 | 1.2 |
| 24 months | 10.9 | 1.1 |
| 30 months | Not determined | 1.1 |

These results clearly show that the solid composition A according to the invention is perfectly stable over a period of at least 24 months, whereas a slow increase in the degraded forms is observed for the liquid composition (more than 5% of degraded forms beyond 6 months at 5° C.).

### Example 6

Comparison of Pharmacokinetic Properties in the Dog of an Aqueous Liquid Composition According to the Invention and a Non-Freeze-Dried Composition

Pharmacokinetic Properties

The liquid composition prepared in Example 5 is injected by sub-cutaneous route into four Beagle dogs weighing approximately 10 kg, in the dorsal position, at a dose of 60 μg of IFN-alpha-2b per kg (i.e. 0.2 mL of composition per kg) in the same study as that described at Example 4. The sampling times and assay methods of the serum samples are identical to those described at Example 4.

The pharmacokinetic parameters corresponding to the liquid composition are shown in Table 7 where they are compared to those obtained with composition B2 in Example 4.

TABLE 7
Pharmacokinetic parameters corresponding to composition B2 and to
liquid composition

| | | Composition B2 |
|---|---|---|
| | liquid | (according to the |
| | composition | invention) |
| Cₘₐₓ ± standard deviation (ng/mL) | 2.0 ± 0.9 | 2.2 ± 0.6 |
| median Tₘₐₓ [interval] (h) | 24 [6-24] | 21 [12-48] |
| AUC₀₋₁₂₀ ± standard deviation | 75.8 ± 33.5 | 121.1 ± 36.5 |
| (ng × h/mL) | | |
| T_{50%AUC} ± standard deviation (h) | 47.3 ± 8.1 | 44.0 ± 5.1 |

Both compositions show similar properties. This indicates that freeze-drying and adding excipients such as sucrose and methionine do not alter the pharmacokinetic properties of the composition.

### Example 7

In Vitro Release Profile of the Compositions According to the Invention

The in vitro release profile of liquid composition B1 according to the invention is determined in an in vitro test using a continuous flow cell. This profile is compared with that of a control solution of IFN-alpha-2b, prepared at 0.3 mg/mL by dilution in phosphate buffered saline, as described in Example 4.

On leaving the cell, the liquid is collected at regular intervals of 30 minutes in the case of composition B1 according to the invention and 2 minutes in the case of the control solution of IFN-alpha-2b.

The cumulated quantity of IFN-alpha-2b collected in the different fractions is indicated in Table 8.

TABLE 8
Cumulated quantity of IFN-alpha-2b assayed over time (expressed in % -
average of 3 tests)

| | | Composition B1 |
|---|---|---|
| Time | Control solution of IFN- | (according to the |
| (h) | alpha-2b | invention) |
| 0 | 0 | 0 |
| 0.5 | 96 | 6 |
| 1 | 99 | 22 |
| 2 | 100 | 47 |
| 3 | 100 | 65 |
| 4 | — | 77 |
| 5.5 | — | 91 |
| 8 | — | 100 |
| 12 | — | 100 |
| 15 | — | 100 |

The results show that 50% of the release is observed in the first minutes in the case of the control solution of IFN-alpha-2b whereas it takes more than 2 hours to release the same quantity of IFN-alpha-2b with the composition B1 according to the invention.

The release profile of the composition B1 is therefore sustained compared with that of a control solution of IFN-alpha-2b.

### Example 8

Preparation and Characterization of an Aqueous Liquid Composition According to the Invention (Composition D1), Reconstituted from a Solid Composition (Composition C) According to the Invention, Containing Polymer P1 and IFN-Alpha-Consensus

Stage 1: Preparation of the Initial Liquid Composition

80.30 g of water for injection, 0.279 g of methionine, 10.81 g of sucrose are added successively to a solution of 133.64 g of polymer P1 at 28.3 mg/mL. The solution obtained is maintained under moderate stirring for 3 hours at ambient temperature then sterilized by filtration on a 0.2 μm filter.

A solution of IFN-alpha-consensus at 0.2 mg/mL (Infergen®) in a buffer containing sodium chloride (100 mM), monobasic sodium phosphate (9.6 mM) and dibasic sodium phosphate (17.4 mM) is concentrated by frontal ultrafiltration (Amicon cell, YM10 membrane, cut-off threshold of 10 kDa) up to a concentration of 3.6 mg/mL then sterilized by filtration on a 0.2 μm filter.

20.67 g of this solution is added to 199.38 g of the solution of polymer P1. The mixture thus obtained is kept under stirring at ambient temperature for 18 hours then sterilized by filtration on a 0.2 μm filter. The solution is then distributed, at a rate of 1.33 g per flask, into 3 mL flasks each containing three 4.76 mm diameter glass beads.

Stage 2: Preparation of a Solid Composition C According to the Invention

The mixture obtained in stage 1, distributed into the flasks, is then freeze-dried in a USIFROID freeze-dryer (model PL 45) with a freeze-drying cycle lasting a total of 72 hours, in order to obtain a solid composition according to the invention.

Stage 3: Reconstitution of an Aqueous Liquid Composition D1 According to the Invention

An injectable aqueous liquid composition D1 is reconstituted by the addition of 0.9 mL of water for injection per flask and stirred manually for a few minutes. The liquid composition D1 has a grafted poly(glutamic acid)/IFN-alpha-consensus weight ratio of 45 and contains 58 mg/mL of sucrose and 1.5 mg/mL of L-methionine.

The characteristics of composition D1 are shown in Table 9.

TABLE 9
Characteristics of the liquid composition D1

| | Composition D1 |
|---|---|
| C_{IFN} (mg/mL) | 0.46 |
| Cₚₒₗ (mg/mL) | 20.6 |
| [polymer P1]/[IFN-alpha-consensus] (g/g) | 45 |
| pH | 7.1 |
| Osmolality (mOsm/kg) | 302 |
| Viscosity (mPa · s) | 22 |
| Hydrodynamic diameter (nm) | 18 |

### Example 9

Measurement of the Percentage of Aggregation of IFN-Alpha-Consensus in a Liquid Composition D1 According to the Invention

A liquid composition D1 according to the invention, containing approximately 0.45 mg/mL of IFN alpha-consensus and 20 mg/mL of polymer P1, is reconstituted as described in stage 3 of Example 8.

A control solution of IFN-alpha-consensus at 0.45 mg/mL is prepared by concentrating by frontal ultrafiltration (Amicon cell, YM10 membrane, cut-off threshold of 10 kDa), up to a concentration of 0.45 mg/mL, a solution of IFN alpha-consensus at 0.2 mg/mL as described in Example 8.

Evaluation of the Aggregation of IFN Alpha-Consensus

i—Visually

Visual inspection shows that these solutions are both clear.

0.9 mL of composition D1 and 0.9 mL of control solution of IFN-alpha-consensus are heated for 1 hour at 90° C.

After this heating, it appears clearly that the composition D1 is still clear whereas the control solution is cloudy, indicating the aggregation of some of the IFN alpha-consensus in the latter.

ii—By UV Absorbance

This difference in behaviour is confirmed by a measurement of the UV absorbance at 450 nm of composition D1 and the control solution of IFN-alpha-consensus on a Perkin Elmer Lambda spectrophotometer 35 equipped with cells with a width of 3 mm, as shown in Table 10.

TABLE 10
UV absorbance at 450 nm of the solutions before and after heating

| | Composition D1 | |
|---|---|---|
| | (according to the | Control solution of IFN- |
| | invention) | alpha-consensus |
| Absorbance before heating | 0.010 | 0.002 |
| (absorbance unit) | | |
| Absorbance after heating | 0.011 | 0.540 |
| (absorbance unit) | | |

The low absorbance of composition D1 after heating indicates that no aggregation of IFN-alpha-consensus occurred.

iii—By Size Exclusion Chromatography

Composition D1 and the control solution of IFN-alpha-consensus before and after heating for 1 hour at 90° C., are analyzed by size exclusion chromatography.

The results are shown in Table 11.

TABLE 11
Percentage of IFN alpha-consensus monomer

| | Composition D1 | Control |
|---|---|---|
| | (according to | solution of IFN- |
| | the invention) | alpha-consensus |
| IFN-alpha-consensus monomer | 100% | 100% |
| peak before heating (surface %) | | |
| IFN-alpha-consensus monomer | 98% | 27% |
| peak after heating (surface %) | | |

These results show that more than 95% of IFN-alpha-consensus is eluted as IFN-alpha-consensus monomer in the case of the heated composition D1, whereas this peak represents no more than 27% in the case of the heated control solution of IFN-alpha-consensus.

### Example 10

Pharmacokinetic Properties in the Dog of an Aqueous Liquid Composition According to the Invention (Composition D2), Containing Interferon Alpha-Consensus, Reconstituted Before Injection from a Solid Composition According to the Invention

Preparation of Composition D2

A liquid composition D2 is prepared as described in Example 8. The liquid composition D2 has a grafted poly(glutamic acid)/IFN-alpha-consensus weight ratio of 44 and contains 53 mg/mL of sucrose and 1.4 mg/mL of L-methionine.

The characteristics of composition D2 are shown in Table 12.

TABLE 11
Characteristics of composition D2

| | Composition D2 |
|---|---|
| C_{IFN} (mg/mL) | 0.45 |
| Cₚₒₗ (mg/mL) | 20.0 |
| [polymer P1]/[IFN-alpha-consensus] (g/g) | 44 |
| pH | 6.4 |
| Osmolality (mOsm/kg) | 340 |
| Hydrodynamic diameter (nm) | 14 |

Pharmacokinetic Properties of Composition D2

Composition D2 is injected by sub-cutaneous route, into three Beagle dogs weighing approximately 13 kg, in the dorsal position, at a dose of IFN-alpha-consensus of 60 μg/kg (i.e. approximately 0.13 mL of solution per kg).

A control solution of IFN-alpha-consensus is prepared at a concentration of 0.2 mg/mL in a solution of phosphate buffered saline (PBS—Sigma) containing 10 mM of monobasic sodium phosphate, 2.7 mM of potassium chloride and 137 mM of sodium chloride. This control solution is also tested in three other Beagle dogs according to a protocol similar to that described above.

The blood samples for determining the plasma concentrations of IFN-alpha-consensus were taken at the following times:
Composition D2: T=0 and 1 hour, 6 hours, 12 hours, 18 hours, 24 hours, 36 hours, 48 hours, 72 hours, 96 hours, 120 hours, 144 hours, 168 hours and 240 hours after injection
Control solution of IFN-alpha-consensus: T=0 and 0.5 hour, 1 hour, 2 hours, 3 hours, 5 hours, 8 hours, 12 hours, 14 hours, 18 hours, 24 hours, 48 hours, 72 hours, 96 hours, 120 hours, 144 hours, 168 hours and 240 hours after injection

The serum samples are frozen at −20° C. Analysis of these samples is then carried out by an ELISA method with a commercial kit (Human IFNα Multi-Subtype ELISA Kit supplied by PBL Interferon Source ref 41105) using IFN-alpha-consensus in a canine serum pool as standard and following the supplier's instructions.

As shown in FIG. 2, the plasma profile of IFN-alpha-consensus released from composition D2 stretches over a period of at least 5 days while the plasma profile of IFN-alpha-consensus released from the control solution cannot be quantified anymore after only one day.

The pharmacokinetic parameters obtained as described in Example 4 are shown in Table 13.

TABLE 13
Pharmacokinetic parameters corresponding to composition D2 and to a
control solution of IFN-alpha-consensus

| | | Composition D2 |
|---|---|---|
| | Control solution | (according to the |
| | of IFN-alpha-consensus | invention) |
| Cₘₐₓ ± standard | 39.7 ± 10.4 | 1.4 ± 0.2 |
| deviation (ng/mL) | | |
| median Tₘₐₓ [interval] (h) | 3 [2-3] | 72 [6-72] |
| AUC₀₋ₜ ± standard | 311.0 ± 49.1* | 127.7 ± 7.6** |
| deviation (ng × h/mL) | | |
| T_{50% AUC} ± standard | 5.1 ± 0.5 | 60.8 ± 6.4 |
| deviation (h) | | |

*AUC calculated between 0 and 48 hours
**AUC calculated between 0 and 240 hours

These results clearly demonstrate, through the reduction in the Cₘₐₓ and the significant extension of the Tₘₐₓ and T_{50% AUC}, the sustained release properties for composition D2 in comparison with the control solution of IFN-alpha-consensus.

### Example 11

Preparation and Characterization of an Aqueous Liquid Composition (Composition F1) Reconstituted from a Solid Composition (Composition E) Containing Polymer P2 and IFN-Alpha-Consensus

Synthesis of Comparative Grafted Poly(Glutamic Acid) Polymer P2

Polymer P2 having an average degree of polymerization of approximately 100 and having a molar grafting rate with alpha-tocopherol of about 5% is synthesized according to a method of preparation similar to that of polymer P1 described in Example 1, but using as starting material alpha-poly(L-glutamic acid) with a DP of approximately 100. Polymer P2 is isolated in the form of aqueous suspension at a concentration of approximately 65 mg/mL.

Preparation of Solid Composition E

A solid composition E is prepared from the previous polymer P2 according to a method of preparation similar to that described in stages 1 and 2 of Example 7 by adjusting the quantity of water added in the first stage to take account of the higher concentration of the initial polymer suspension.

Reconstitution of an Aqueous Liquid Composition F1

An aqueous liquid composition F1 is reconstituted by adding water to the solid composition E, as described in stage 3 of Example 8. The liquid composition F1 has a grafted poly(glutamic acid)/IFN-alpha-consensus weight ratio of 42 and contains 53 mg/mL of sucrose and 1.4 mg/mL of L-methionine.

The characteristics of composition F1 are shown in Table 14.

TABLE 14
Characteristics of liquid composition F1

| | Composition F1 |
|---|---|
| C_{IFN} (mg/mL) | 0.45 |
| Cₚₒₗ (mg/mL) | 19.0 |
| [polymer P2]/[IFN-alpha-consensus] (g/g) | 42 |
| pH | 6.3 |
| Osmolality (mOsm/kg) | 321 |
| Hydrodynamic diameter (nm) | 13 |

### Example 12

Pharmacokinetic Properties in the Dog of an Aqueous Liquid Composition (Composition F1) of IFN Alpha-Consensus Associated to a Polymer P2

The composition F1 is injected by sub-cutaneous route into three Beagle dogs weighing from 11 to 13 kg, in the dorsal position, at a dose of IFN-alpha-consensus of 60 μg/kg in the same study as that described in Example 10. The samples are taken and the assays are carried out in the same manner.

The pharmacokinetic parameters obtained are shown in Table 15.

TABLE 15
Pharmacokinetic parameters corresponding to composition F1

| | Composition F1 |
|---|---|
| Cₘₐₓ ± standard deviation (ng/mL) | 8.3 ± 0.9 |
| median Tₘₐₓ [interval] (h) | 6 [6-18] |
| AUC₀₋ₜ* ± standard deviation (ng × h/mL) | 200.3 ± 7.5 |
| T_{50% AUC} ± standard deviation (h) | 18.5 ± 2.9 |

*AUC calculated between 0 and 240 hours

The results show clearly that the Tₘₐₓ and the T_{50% AUC} for composition Fl are lower than those obtained for the composition D2 of Example 10. This therefore indicates a faster release of the IFN-alpha-consensus with composition F1, thus less advantageous for a once-a-week administration.

### Example 13

Comparison of Pharmacokinetic Properties in the Dog of Two Aqueous Liquid Compositions of IFN-Alpha-Consensus Containing Respectively Polymer P1 (Composition D2) and Polymer P2 (Composition F2)

An aqueous liquid composition F2 is prepared by the addition of water to the solid composition E, in order to obtain a polymer concentration of 35 mg/mL. Liquid composition F2 has a grafted poly(glutamic acid)/IFN-alpha-consensus weight ratio of 68.6 and contains 44.6 mg/mL of sucrose and 1.14 mg/mL of L-methionine.

The characteristics of composition F2 are shown in Table 16.

TABLE 16
Characteristics of liquid composition F2

| | Composition F2 |
|---|---|
| C_{IFN} (mg/mL) | 0.51 |
| Cₚₒₗ (mg/mL) | 35.0 |
| [polymer P2]/[IFN-alpha-consensus] (g/g) | 68.6 |
| pH | 6.5 |
| Osmolality (mOsm/kg) | 318 |
| Hydrodynamic diameter (nm) | 14 |

Pharmacokinetic Properties

Composition F2 is injected by sub-cutaneous route, into three Beagle dogs weighing from 11 to 14 kg, at a dose of IFN-alpha-consensus of 60 μg/kg in the same study as that described in Example 10. The samples are taken and the assays are carried out in the same manner.

The pharmacokinetic parameters obtained with composition F2 are compared to those obtained with composition D2 in Table 17.

TABLE 17
Pharmacokinetic parameters corresponding to composition F2

| | Composition D2 | |
|---|---|---|
| | (according to the | |
| | invention) | Composition F2 |
| Cₘₐₓ ± standard deviation (ng/mL) | 1.4 ± 0.2 | 3.6 ± 2.9 |
| median Tₘₐₓ [interval] (h) | 72 [6-72] | 24 [6-36] |
| AUC_{0-24 h} ± standard | 127.7 ± 7.6 | 118.9 ± 49.1 |
| deviation (ng × h/mL) | | |
| T_{50% AUC} ± standard deviation (h) | 60.8 ± 6.4 | 32.6 ± 7.5 |

These results clearly demonstrate that the Tₘₐₓ and T_{50% AUC} of composition F2 are lower than those obtained with composition D2 in Example 10.

This indicates a faster release of IFN-alpha-consensus with composition F2, thus less advantageous for a once-a-week administration.

### Example 14

Preparation and characterization of an aqueous liquid composition (composition H) reconstituted from a solid composition (composition G), containing polymer P3 and IFN-alpha-2b

Synthesis of comparative grafted poly(glutamic acid) polymer P3 Polymer P3 having an average degree of polymerization of approximately 100 and having a molar grafting rate with alpha-tocopherol of about 20% is synthesized according to a method of preparation similar to that of polymer P1 described in Example 1, but using as starting material alpha-poly(L-glutamic acid) with a DP of approximately 100. Polymer P3 is isolated in the form of aqueous suspension at a concentration of approximately 79 mg/mL.

Stage 1: Preparation of the Initial Liquid Composition

4.34 g of water for injection, 0.04 g of a 1 N soda solution, 1.54 g of a sucrose solution at 300 mg/g and 0.33 g of a methionine solution at 40 mg/g are added successively to a solution of 5.76 g of polymer P3 at 79 mg/mL. The solution of polymer P3 obtained is maintained under moderate stirring for 2 hours at 25° C.

A frozen solution of IFN-alpha-2b at 2.04 mg/mL (BioSidus—Argentine) in a buffer containing citric acid (25 mM), dibasic sodium phosphate (50 mM) and sodium chloride (150 mM) is thawed at ambient temperature.

1.3 g of this solution is added to 11.88 g of the solution of polymer P3. The mixture thus obtained is left under stirring at 25° C. for 2 hours then left under stirring for another 16 hours at 25° C. The mixture is then divided, at a rate of 1.31 g per flask, into 3 mL flasks each containing 3 glass beads with a diameter of 4.76 mm.

Stage 2: Preparation of the Solid Composition G

The mixture obtained in stage 1, divided into the flasks, is then lyophilized in a USIFROID freeze-dryer with a freeze-drying cycle lasting a total of 87 hours, in order to obtain the solid composition G.

Stage 3: Reconstitution of an Aqueous Liquid Composition H

The aqueous liquid composition H is reconstituted by the addition of 0.85 mL of water for injection to the solid composition G. The liquid composition H has a grafted poly(glutamic acid)/IFN-alpha-2b weight ratio of 166.7 and contains 51.9 mg/mL of sucrose and 1.5 mg/mL of L-methionine.

The characteristics of composition H are shown in Table 18.

TABLE 18
Characteristics of liquid composition H

| | Composition H |
|---|---|
| C_{IFN} (mg/mL) | 0.3 |
| Cₚₒₗ (mg/mL) | 50.0 |
| [polymer P3]/[IFN-alpha-2b] (g/g) | 166.7 |

### Example 15

Comparison of In Vitro Release Profiles of Two Aqueous Liquid Compositions of IFN-Alpha-2b Containing Respectively Polymer P1 (Composition B3) and Polymer P3 (Composition H)

The in vitro release profile of liquid composition H out of the scope of the invention is determined in the in vitro test T2 by means of a continuous flow cell. This profile is compared to the profile of a liquid composition B3 according to the invention obtained according to a method of preparation similar to that described in Example 2.

The cumulated IFN-alpha-2b amount collected in to various fractions is shown in Table 19.

TABLE 19
Cumulated amount of IFN-alpha-2b assayed as a function of time (in % -
average of 3 assays)

| | | Composition B3 |
|---|---|---|
| Time | | (according to the |
| (min) | Composition H | invention) |
| 0 | 0 | 0 |
| 20 | 28.1 | 9.0 |
| 50 | 72.4 | 33.0 |
| 80 | 89.6 | 47.9 |
| 110 | 96.3 | 58.0 |
| 140 | 98.5 | 65.1 |
| 170 | 99.2 | 71.1 |
| 200 | 99.5 | 76.0 |
| 230 | 99.7 | 80.4 |
| 260 | 99.7 | 84.5 |
| 320 | 99.8 | 91.0 |
| 440 | 99.9 | 97.9 |
| 680 | 100.0 | 99.9 |
| 890 | 100.0 | 100.0 |

The results show that 60% of the IFN-alpha-2b are released in less than 50 minutes from liquid composition H, out of the scope of the invention while more than 110 minutes are necessary to release the same amount of IFN-alpha-2b from composition B3 according to the invention.

Composition B3 according to the invention is characterized by a slower release of IFN-alpha-2b, thus more advantageous for a once-a-week administration.

### Example 16

Comparison of Pharmacokinetic Properties in the Rat of an Aqueous Liquid Compositions of IFN-Alpha-2b Containing Respectively Polymer P1 (Composition I) and Polymer P3 (Composition J)

Preparation of Liquid Composition I

2.25 g of water for injection, 0.66 g of a methionine solution at 40.4 mg/g, 80 μL of a 1 N soda solution and 0.51 g of a sodium chloride solution at 20% are added successively to a solution of 11.54 g of polymer P1 at 30.9 mg/mL.

A solution of IFN-alpha-2b at 2 mg/mL (BioSidus—Argentine) is provided in a buffer containing citric acid (25 mM), dibasic sodium phosphate (50 mM) and sodium chloride (150 mM).

1.49 g of this solution is added to 8.53 g of the polymer P1 solution. The mixture thus obtained is left under stirring at 25° C. overnight.

The characteristics of liquid composition I are shown in Table 20.

TABLE 20
Characteristics of liquid composition I

| | Composition I |
|---|---|
| C_{IFN} (mg/mL) | 0.3 |
| Cₚₒₗ (mg/mL) | 20 |
| [polymer P1]/[IFN-alpha-2b] (g/g) | 66.7 |

Preparation of Liquid Composition J

0.12 g of water for injection, 24 μL of a 1 N soda solution and 8 μL of a sodium chloride solution at 20% are added successively to a solution of 0.90 g of polymer P3 at 209 mg/mL.

A solution of IFN-alpha-2b at 2 mg/mL (BioSidus—Argentine) is provided in a buffer containing citric acid (25 mM), dibasic sodium phosphate (50 mM) and sodium chloride (150 mM).

0.13 g of this solution is added to 0.74 g of the polymer P1 solution. The mixture thus obtained is left under stirring at 25° C. overnight.

The characteristics of this reference composition are shown in Table 21.

TABLE 21
Characteristics of composition J

| | Composition J |
|---|---|
| C_{IFN} (mg/mL) | 0.3 |
| Cₚₒₗ (mg/mL) | 153 |
| [polymer P3]/[IFN-alpha-2b] (g/g) | 510 |

Pharmacokinetic Properties

Compositions I and J injected by sub-cutaneous route, into eight (four in each sub-group) rats Sprague-Dawley, at a dose of IFN-alpha-2b of 300 μg/kg. The blood samples for determining the plasma concentrations of IFN-alpha-2b are taken alternatively in each sub-group at the following times: T=0 hours, 3 hours, 6 hours, 12 hours, 24 h, 48 hours, 72 hours, 96 hours, 120 hours, and 168 hours after injection.

The serum samples are frozen at −80° C. Analysis of these samples is then carried out by an ELISA method as described in Example 4.

The pharmacokinetic parameters obtained are shown in Table 22.

TABLE 22
Pharmacokinetic parameters

| | Composition I | Composition J |
|---|---|---|
| Cₘₐₓ (ng/mL) | 9.2 | 0.5 |
| AUC_{0-168 h} (ng × h/mL) | 197 | 30 |

Composition J comprises polymer P3 carrying grafts of alpha-tocopherol at a molar grafting rate of 20% and has a polymer P3/IFN-alpha weight ratio of 510. This composition corresponds neither to the criterion of the grafting rate with alpha-tocopherol nor to the criterion of the polymer/IFN-alpha weight ratio of the invention.

The bioavailability obtained with composition J, expressed by AUC_{0-168 h}, is much lower than that obtained with composition I comprising polymer P1 which has a grafting rate with alpha-tocopherol and a polymer/IFN-alpha weight ratio satisfying the criteria of the invention.

The results show that when the grafted poly(glutamic acid) concentration and the grafting rate with alpha-tocopherol are too high, the bioavailability of IFN-alpha is therefore reduced.

### Example 17

Clinical Evaluation of Composition K Compared to Viraferon®Peg: Pharmacokinetic, Antiviral Activity and Tolerance

Assayed liquid composition K contains IFN-alpha-2b at a concentration of 0.29 mg/mL, polymer P1 at a concentration of 22.3 mg/ml and sodium chloride at a concentration of 8.1 mg/mL.

The characteristics of composition K are shown in Table 23.

TABLE 23
Characteristics of composition K

| | Composition K |
|---|---|
| C_{IFN} (mg/mL) | 0.29 |
| Cₚₒₗ (mg/mL) | 22.3 |
| [polymer P1]/[IFN-alpha-2b] (g/g) | 77 |
| pH | 6.4 |
| Osmolality (mOsm/kg) | 296 |
| Viscosity (mPa · s) | 10 |

In a multicenter, randomized, open-label, clinical trial liquid composition K was compared to the marketed reference product (Viraferon®Peg, Schering-Plough). This study included 2 successive injections, within a week of each other, by sub-cutaneous route, of composition K at a dose of 27 million units per week or of Viraferon®Peg at a dose of 1.5 μg/kg/week (12 and 14 patients respectively) to naive, non-responder or relapser patients affected by viral hepatitis C.

Blood serum samples were taken before the second injection (t₀), then 1, 6, 12, 18, 24, 36, 48, 72, 96, 120 and 168 hours after the second injection. Analysis of the IFN-alpha-2b concentration in the samples was carried out by an ELISA method (Amersham High Sensitivity Interferon-Alpha [(h)IFNα] Human, Biotrak ELISA System).

Viral concentrations were obtained via a quantitative assay of hepatitis C virus RNA by a PCR technique (Bayer Versant® HCV RNA 3.0).

The following pharmacokinetic and pharmacodynamic parameters were calculated using the WinNonlin software non-compartmental analysis (NCA) method (Pharsight Corporation):
Cₘₐₓ, Tₘₐₓ: the average maximum serum concentration Cₘₐₓ is the average of the Cₘₐₓ of each patient and the median time Tₘₐₓ is the the median of the Tₘₐₓ of each patient, obtained by direct reading of the concentration points;
AUC₀₋ₜ: the area under the curve of concentration as a function of time was calculated between the time t₀ and the last time for which concentration can be quantified, using the trapezoid rule;
CVₘᵢₙ, Tₘᵢₙ: the average minimum viral load CVₘᵢₙ is the average of the CVₘᵢₙ of each patient and the median time Tₘᵢₙ is the the median of the Tₘᵢₙ of each patient, obtained by direct reading of the concentration points;
AUCv: the area under the curve of viral load as a function of time was calculated between the first and last time below the baseline, using the trapezoid rule,
CV₁₆₈ₕ: the viral load at 168 hours is the average of the CV₁₆₈ₕ of each patient, obtained by direct reading of the concentration points.

Analysis of the antiviral activity was carried out in each group in the 9 patients of genotype 1 who constitute a homogenous sub-population which is representative of the majority of patients affected by viral hepatitis C and who are also harder to treat.

The assessment of safety and tolerance was based on the collection of undesirable events reported either spontaneously or by the clinical examination and free questioning of patients, as well as the assessment of vital signs, electrocardiographic recording, and biological testing results.

The assessment of local tolerance on the whole population was carried out by examining the injection site (semi-quantitive assessment of erythema, heat (inflammation), edema, induration, pruritis, pain) before injection, then 6 hours after, and daily during the 7 days following the injection.

Pharmacokinetic Results

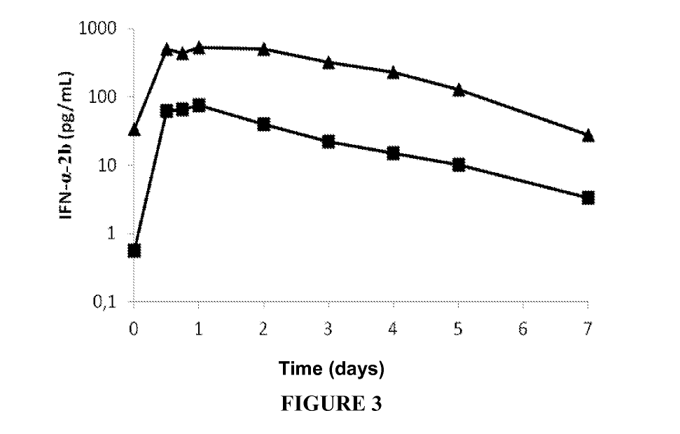

The pharmacokinetic profile and the pharmacokinetic parameters obtained with composition K and Viraferon®Peg in respectively 10 and 13 patients are represented in FIG. 3 and in Table 24 respectively.

TABLE 24
Pharmacokinetic parameters corresponding to composition K and
Viraferon ® Peg

| | Viraferon ® Peg | Composition K |
|---|---|---|
| | (n = 13) | (n = 10) |
| Cₘₐₓ ± standard deviation | 631 ± 332 | 81.8 ± 44.8 |
| (pg/mL) | | |
| median Tₘₐₓ [interval] (h) | 24 [0-73] | 24 [12-72] |
| AUC₀₋ₜ ± standard | 47368.0 ± 20193.8 | 4274.2 ± 1992.1 |
| deviation (pg × h/mL) | | |

Both compositions show a median Tₘₐₓ of 24 hours and a persistence of circulating IFN-alpha-2b during 1 week after administration. The superior amount of circulating interferon from Viraferon®Peg is due to the pegylated form of IFN-alpha-2b which slows downs its elimination, but has no influence on the efficacy. Composition K has a good efficacy as shown in the remainder or the example.

Antiviral Activity

The pharmacodynamic parameters obtained are shown in Table 25.

TABLE 25
Pharmacodynamic parameters corresponding to composition K and
Viraferon ® Peg obtained in patients of génotype 1

| | Viraferon ® Peg | Composition K |
|---|---|---|
| | (n = 9) | (n = 9) |
| CVₘᵢₙ ± standard deviation | −1.07 ± 0.79 | −1.03 ± 0.54 |
| Log(CV)(IU/mL) | | |
| median Tₘᵢₙ [interval] (h) | 36 [12-120] | 48 [0-120] |
| AUCv ± standard deviation | 120.6 ± 138.4 | 156.4 ± 106.3 |
| (IU/mL × hr) | | |
| CV_{168 h} ± standard deviation | −0.21 ± 0.32 | −0.57 ± 0.49 |
| Log(CV)(IU/mL) | | |

After the second administration, the antiviral pressure exerted on the infection by the compositions (represented by AUCv) is stronger for composition K than for

Viraferon®Peg (respectively 156 and 121 IU/mL×h). Besides, the viral load at 168 hours is statistically lower for composition K than for Viraferon®Peg (respectively −0.57 and −0.21 log, unilateral Student test p<0.05). Thus, composition K at a dose of 27 million units per week provides an antiviral activity superior to that of the reference treatment Viraferon®Peg at a dose of 1.5 μg/kg/week, for much lower plasma concentrations of IFN-alpha-2b. This indicates excellent antiviral activity of circulating IFN-alpha-2b after injecting composition K.

Safety and Tolerance

The list of undesirable events reported in each f the two groups is presented in Table 26 below.

TABLE 26
List of undesirable events possibly, probably or certainly related to
composition K or Viraferon ® Peg

| | Composition K | Viraferon ® Peg |
|---|---|---|
| Nature of the undesirable event | (n = 12) | (n = 14) |
| General disorders and injection | 31 | 57 |
| site reactions, including | | |
| Injection site reactions | 20 | 44 |
| Asthenia | 5 | 1 |
| Influenza-like illness | 1 | 1 |
| Irritability | 1 | 0 |
| Fever | 4 | 11 |
| Central nervous system disorders, | 14 | 14 |
| including | | |
| Headaches | 13 | 14 |
| Olfactory disorders | 1 | 0 |
| Blood or lymphatic system | 7 | 16 |
| disorders, including | | |
| Leukopenia/Lymphopenia/ | 6 | 16 |
| Neutropenia | | |
| Thrombocytopenia | 1 | 0 |
| Musculoskeletal disorders, including | 5 | 13 |
| Joint pain | 0 | 1 |
| Muscle stiffness | 5 | 11 |
| Torticollis | 0 | 1 |
| Ocular abnormality, including | 1 | 1 |
| Conjunctival irritation | 0 | 1 |
| Eye pain | 1 | 0 |
| Gastrointestinal disorders, including | 1 | 2 |
| Nausea | 1 | 1 |
| Vomiting | 0 | 1 |
| Cutaneous or subcutaneous tissue | 1 | 1 |
| anomalies, including | | |
| Pruritus | 0 | 0 |
| Seborrheic dermatitis | 1 | 0 |
| Excessive sweating | 0 | 1 |
| Infectious and parasitic diseases | 1 | 0 |
| (Influenza) | | |
| Biological testing (Urinary | 0 | 1 |
| Ketone bodies) | | |
| Metabolic disorders | 1 | 0 |
| (Hypertriglyceridemia) | | |
| Psychiatric disorders (Anxiety) | 0 | 1 |
| Respiratory, Thoracic and | 1 | 0 |
| Mediastinal Disorders (Bronchial | | |
| congestion) | | |
| Total number of events | 63 | 106 |
| Number of events/patient | 5.2 | 7.6 |

Composition K presented less undesirable events per patient as compared to Viraferon®Peg (respectively 5.3 and 7.6 undesirable events per patient), with notably less systemic undesirable events related to the treatment (fever and blood or lymphatic system disorders) and a decrease by half of local reactions at the injection site.

In conclusion, after the 2 successive injections, within a week of each other, of composition K (at a dose of 27 million units per week) or of Viraferon®Peg (at a dose of 1.5 μg/kg/week), to patients affected by viral hepatitis C, composition K showed:
a pharmacokinetic profile perfectly adapted for a weekly administration;
antiviral properties superior to that of the reference product, providing to composition K a promising therapeutic potential, evidenced by a stronger antiviral pressure and a viral load at 168 hours statistically inferior to that of Viraferon®Peg;
a tolerance profile similar, but with a substantial decrease of the number of undesirable events per patient, particularly fever and blood disorders, as well as a decrease by half of reactions at the injection site. These undesirable events being the main causes of dose reduction or even premature interruption of the interferon treatment, composition K should allow a better compliance and therefore a better efficacy in the case of a prolonged treatment.

1. Solid composition comprising at least one interferon alpha (IFN-alpha) and at least one grafted poly(glutamic acid) having an average molar mass ranging from 26,000 to 40,000 g/mol and carrying grafts of alpha-tocopherol at an average molar grafting rate ranging from 4.5 to 5.5%, characterized in that the IFN-alpha and said grafted poly(glutamic acid) are present in a grafted poly(glutamic acid)/IFN-alpha weight ratio ranging from 21 to 125.

2. Composition according to claim 1, characterized in that the IFN-alpha is chosen from IFN-alpha-2b and IFN-alpha-consensus.

3. Composition according to any one of the previous claims, characterized in that said grafted poly(glutamic acid) is a grafted alpha-poly(glutamic acid)-

4. Composition according to any one of the previous claims, characterized in that said grafted poly(glutamic acid) has an average molar grafting rate in grafts of alpha-tocopherol of approximately 5%.

5. Composition according to any one of the previous claims, characterized in that it comprises at least one antioxidant in an antioxidant/IFN-alpha weight ratio ranging from 2 to 7.

6. Composition according to any one of the previous claims, characterized in that it comprises at least one lyoprotective excipient in a lyoprotective excipient/IFN-alpha weight ratio ranging from 50 to 500.

7. Composition according to claim 2, characterized in that the IFN-alpha is IFN-alpha-2b.

8. Composition according to the previous claim, characterized in that the IFN-alpha-2b and said grafted poly(glutamic acid) are present in a grafted poly(glutamic acid)/IFN-alpha-2b weight ratio ranging from 54 to 100.

9. Composition according to one of claim 7 or 8, characterized in that it comprises methionine in a methionine/IFN-alpha-2b weight ratio ranging from 4 to 6.4.

10. Composition according to any one of claims 7 to 9, characterized in that it comprises at least one sugar in a sugar/IFN-alpha-2b weight ratio ranging from 110 to 280.

11. Solid composition according to any one of claims 7 to 10, characterized in that it comprises the following constituents:
a) approximately 0.3 mg of IFN-alpha-2b;
b) approximately 22 mg of said grafted poly(glutamic acid);
c) approximately 1.5 mg of methionine; and
d) approximately 53 mg of sucrose;
or a multiple or sub-multiple of said quantities.

12. Composition according to claim 2, characterized in that the IFN-alpha is IFN-alpha-consensus.

13. Composition according to the previous claim, characterized in that the IFN-alpha-consensus and said grafted poly(glutamic acid) are present in a grafted poly(glutamic acid)/IFN-alpha-consensus weight ratio ranging from 30 to 60.

14. Composition according to one of claim 12 or 13, characterized in that it comprises methionine in a methionine/IFN-alpha-consensus weight ratio ranging from 2.5 to 3.5.

15. Composition according to any one of claims 12 to 14, characterized in that it comprises at least one sugar in a sugar/IFN-alpha-consensus weight ratio ranging from 70 to 160.

16. Solid composition according to any one of claims 12 to 15, characterized in that it comprises the following constituents in the proportions indicated:
a) approximately 0.45 mg of IFN-alpha-consensus;
b) approximately 20 mg of said grafted poly(glutamic acid);
c) approximately 1.5 mg of methionine; and
d) approximately 58 mg of sucrose;
or a multiple or sub-multiple of said quantities.

17. Composition according to any one of the previous claims, characterized in that it is stable at 5° C. for at least 24 months.

18. Composition according to any one of the previous claims, characterized in that its percentage of IFN-alpha in the monomeric form is greater than 80%, preferably greater than 90%, preferably even greater than 95% of the total quantity of IFN-alpha.

19. Solid pharmaceutical composition based on IFN-alpha, comprising a solid composition as defined according to any one of claims 1 to 18.

20. Use of a solid pharmaceutical composition according to claim 19 for the preparation of a liquid, in particular an injectable, pharmaceutical composition.

21. Aqueous liquid composition comprising at least one IFN-alpha and at least one grafted poly(glutamic acid) having an average molar mass ranging from 26,000 to 40,000 g/mol and carrying grafts of alpha-tocopherol at an average molar grafting rate ranging from 4.5 to 5.5%, the IFN-alpha and said grafted poly(glutamic acid) being present in a grafted poly(glutamic acid)/IFN-alpha weight ratio ranging from 21 to 125, characterized in that it is obtained by the addition of an aqueous liquid to a solid composition as defined according to any one of claims 2 to 16.

22. Composition according to the previous claim, characterized in that its IFN-alpha concentration is comprised between 0.2 and 0.8 mg/mL and its grafted poly(glutamic acid) concentration is comprised between 17 and 25 mg/mL.

23. Composition according to one of claim 21 or 22, characterized in that the IFN-alpha is IFN-alpha-2b and in that its IFN-alpha-2b concentration is comprised between 0.27 and 0.33 mg/mL.

24. Composition according to one of claim 21 or 22, characterized in that the IFN-alpha is IFN-alpha-consensus and in that its IFN-alpha-consensus concentration is comprised between 0.40 and 0.50 mg/mL.

25. Composition according to any one of claims 21 to 24, characterized in that the aqueous liquid is water, in particular water for injection.

26. Composition according to any one of claims 21 to 25, characterized in that it is presented in the form of an aqueous suspension of hydrogels, of nanometric size, in particular with an average hydrodynamic diameter by volume comprised between 10 and 60 nm.

27. Composition according to any one of claims 21 to 26, characterized in that it has a pH comprised between 6.3 and 7.4.

28. Composition according to any one of claims 21 to 27, characterized in that it has an osmolality comprised between 270 and 350 mOsmol.

29. Composition according to any one of claims 21 to 28, characterized in that it has a viscosity, measured at 20° C. and at a shear rate of 10 s⁻¹, less than 1,000 mPa·s, in particular less than 500 mPa·s, more particularly comprised between 5 and 200 mPa·s.

30. Method for the preparation of a solid composition according to any one of claims 1 to 11 or 17 to 18, comprising the following stages:
(a) providing an aqueous liquid solution of grafted poly(glutamic acid) in a concentration comprised between 20 and 30 mg/g;
(b) providing a solution of IFN-alpha-2b in a concentration comprised between 1.5 and 2.7 mg/mL, preferably in a concentration of approximately 2.1 mg/mL; with at least one component, selected from a lyoprotective excipient, an antioxidant and a pH adjustment excipient, being present in at least one of the solutions of stage (a) or of stage (b);
(c) mixing the solutions of stages (a) and (b) so that after mixing, the composition obtained:
has a grafted poly(glutamic acid) concentration comprised between 10 and 15 mg/g,
has an osmolality comprised between 130 and 230 mOsm/kg,
has a pH comprised between 6.2 and 6.8, preferably of 6.5;
(d) subjecting said mixture to at least one sterilization operation; and
(e) dehydrating the solution in order to form said solid composition.

31. Method for the preparation of a solid composition according to any one of claims 1 to 6 or 12 to 18, comprising the following stages:
(a) providing a grafted poly(glutamic acid) solution in a concentration comprised between 20 and 30 mg/g;
(b) providing an IFN-alpha-consensus solution in a concentration comprised between 2.0 and 5.0 mg/mL; with at least one component, selected from a lyoprotective excipient, an antioxidant and a pH adjustment excipient, being present in at least one of the solutions of stage (a) or of stage (b);
(c) mixing the solutions of stages (a) and (b) so that after mixing, the composition :
has a grafted poly(glutamic acid) concentration comprised between 10 and 15 mg/g;
has an osmolality comprised between 130 and 250 mOsm/kg, preferably 225 mOsm/kg,
has a pH comprised between 6.8 and 7.2, preferably 7.0;
(d) subjecting said mixture to at least one sterilization operation; and
(e) dehydrating the solution in order to form said solid composition.
